(19) United States
(12) Patent Application Publication
(10) Pub. No.: US 2005/0143282 A1
(43) Pub. Date: Jun. 30, 2005

(54) Emulsions

(76) Inventors: Serge Creutz, Rocourt (BE); Houria Seghir, Nivelles (BE); Guzman Nachon, Barcelona (ES); Bertrand Lenoble, Hellebecq (BE); Bertrand Michel, Nivelles (BE)

Correspondence Address: DOW CORNING CORPORATION CO1232, 2200 W. SALZBURG ROAD, P.O. BOX 994, MIDLAND, MI, 48686-0994, US

(21) Appl. No.: 10/507,647

(22) PCT Filed: Mar. 27, 2003
(86) PCT No.: PCT/EP03/03309

(30) Foreign Application Priority Data
Apr. 3, 2002 (GB) 0207647.9

(51) Int. Cl. A61K 7/46

(52) U.S. Cl. 512/4

(57) ABSTRACT

An emulsion for controlled fragrance release comprises a disperse phase which is a blend of a fragrance composition and a waxy hydrophobic material having a melting point in the range 10-200° C. dispersed in a continuous phase comprising an aqueous solution of concentration at least 0.1 molar of a salt capable of ionic disassociation in water.

This invention relates to fragrance compositions and to the incorporation of fragrance compositions in cleaning products such as laundry detergents, household cleaning products including hard surface cleaners and polishes, fabric softeners, hair shampoos and soaps and shower gels for personal washing, in other personal products such as antiperspirants or deodorants, or in air fresheners or tumble drier sheets.

Fragrances are frequently incorporated in detergents and other cleaning products to give a pleasant odour during use of the cleaning product and to mask the inherent smell of the soap or other surfactant present in the cleaning product The fragrances are generally complex mixtures of fragrant compounds of varying volatility. Upon storage in a cleaning composition, perfumes and fragrances can be altered through interactions and/or reactions with the other components of the composition. Due to their volatile nature, the fragrant compounds tend to be dissipated with time. Moreover, when used, such as during washing of fabrics with a laundry detergent, most of the perfume is also lost in the aqueous phase during the washing cycle. It has been recognised as desirable that the fragrance should survive storage in the cleaning composition and also survive the cleaning process and should be deposited on the fabric, so that fabrics laundered with a detergent containing the fragrance should have the pleasant odour of the fragrance.

Furthermore, once adsorbed onto the targeted surface, for example fabrics or hair or skin, the fragrance tends to be dissipated very quickly. There is thus a need to improve the storage stability of perfumes and fragrances, their delivery in the application and their long-lasting effect.

Various methods of protecting the fragrance composition have been proposed. The perfume may be mixed with a porous carrier such as zeolite and then coated with a protective barrier, for example a sugar derivative before incorporation in a laundry detergent as described in WO98/41607. U.S. Pat. No. 4,973,422 describes encapsulating perfume particles with apH sensitive coating comprising an acrylic resin and cellulose esters. WO-A-98/28936 describes mixing the perfume with an aqueous slurry of polymer beads made of hydrophobic polyacrylate; polyvinyl alcohol can be adsorbed at the surface of the beads to improve deposition. WO-A-00/02981 describes reacting a perfume component with an amine to obtain a release of the active component over a longer period of time.

U.S. Pat. No. 6,050,129 relates to a process for testing diffusivity, odour character and odour intensity of a fragrance material used in an air freshener and describes mixing perfume with a hydrophobic wax such as candelilla wax or camauba wax and emulsifying the blend in water, preferably with cationic surfactants, to form a long lasting fragrance composition for use in a hair care composition such as a shampoo/conditioner.

WO-A-01/25389 describes a domestic care product comprising a fragrance particle. The particle comprises a fragrance composition and a silicone polymer having a melting point of at least 10° C. At least 20% of the silicone atoms in the silicone polymer have a substituent of 16 carbon atoms or more.

GB-A-1587122 describes fabric conditioning compositions containing particles comprising a perfume and a water-insoluble non-cationic organic carrier, e.g. a wax.

EP-A-539025 describes spray dried composite fragrance microcapsules for fabric conditioning comprising particles of an emulsifiable mixture of wax material and a perfume composition.

U.S. Pat. No. 5,506,201 describes producing fragrance-containing solid particles for incorporation into laundry detergent by melting a fat component such as a fatty acid glyceride, melting a nonionic surfactant with an HLB of 4.3 to 8.6 and combining the melts with an aroma chemical to form a mixture which is rapidly cooled to form a solid material.

EP-A-908174 describes a fragrance composition comprising ellipsoidal hydrophobic particles consisting of a single phase solid solution of a hydrophobic polymer or wax of melting point 35-120° C. having dissolved therein. a hydrophobic fragrance material, and a hydrophilic surfactant proximate to the outer surface of the particles.

According to the present invention an emulsion comprises a disperse phase which is a blend of a fragrance composition and a waxy hydrophobic material having a melting point in the range 10-200° C. dispersed in a continuous phase comprising an aqueous solution of concentration at least 0.1 molar of a salt capable of ionic disassociation in water.

When perfumes or fragrances are solubilised or dispersed in hydrophobic matrices as described in WO-A-01/25389, we have found that the efficiency of loading, and hence of controlled release of the perfume or fragrance, is very dependent on the partition coefficient between the continuous phase and the hydrophobic matrix. We have found that this efficiency can be kept high by increasing the ionic strength of the continuous phase, so that the perfume tends to stay in the wax phase rather than diffusing into the continuous phase. Increasing the ionic strength of the continuous phase also acts as a protective barrier which improves the storage stability of the emulsion and of compositions containing it.

The fragrance composition may be solid or liquid and may be a single fragrant compound, or a natural scented oil, or may be a mixture of fragrant compounds and/or natural oils. Examples of such natural oils and fragrant compounds are described in WO-A-01/25389. The fragrance composition can alternatively comprise a chemicallyprotected fragrance compound such as a reaction product of the fragrance compound.

The waxy hydrophobic material which is mixed with the fragrance composition to form the disperse phase is preferably a polymer and is most preferably a polysiloxane containing hydrocarbon substituents having 12 or more carbon atoms. The polysiloxane is preferably a polydiorganosiloxane comprising methyl alkyl siloxane units ((CH3)(R′)SiO_{2/2}), where R′ is a long chain alkyl group having 12 or more, preferably 16 to 100 carbon atoms, optionally together with dimethyl siloxane units or units of the formula ((CH₃)R′)SiO₂) where R″ is an alkyl group having 1-11 carbon atoms, for example ethyl, a cycloalkyl group such as 2-cyclohexylethyl, a haloalkyl group, an aryl group such as phenyl or an aralkyl group such as 2-phenylpropyL 2-phenylethyl or 2-(t-butylphenylethyl). The methyl group of the above siloxane units could be replaced by ethyl or another lower alkyl group if desired. The long chain alkyl group R′ can optionally be substituted by polar substituents such as amino, amido, alcohol, alkoxy, or ester groups. Preferably at least 20% of the silicon atoms in the polysiloxane, and most preferably at least 50%, have an alkyl substituent having 16 to 100 carbon atoms, most preferably 20 to 36 carbon atoms. The polysiloxane may be linear or may be branched, for example it may contain CH3 SiO3/2 units or R′SiO_{3/2} units. Altematively the polysiloxane can be cyclic, for example a cyclic polysiloxane containing 4 or 5 methyl alkyl siloxane units in which the said alkyl group has 16 to 100, most preferably 20 to 36, carbon atoms.

The waxy hydrophobic material can alternatively be an organic wax containing no silicon atom such as microcrystalline wax, paraffin wax or a mixture thereof, a long chain fatty acid or a waxy ester thereof such as a triglyceride, for example glyceryl tristearate, a monoester such as octadecyl hexadecanoate, a diester such as ethylene glycol distearate or a tetraester such as pentaeryhthritol tetrastearate, or a long chain fatty alcohol, a long chain fatty amine, a long chain fatty amide, an ethoxylated fatty acid or fatty alcohol, a long chain alkyl phenol or polyethylene wax. In general the long chain of the fatty acid, alcohol, amine or amide is an alkyl group of at least 12 and preferably at least 16 carbon atoms.

The waxy hydrophobic material can be a blend of a wax, particularly an organic wax, with a liquid silicone, for example a polydiorganosiloxane, a branched liquid polysiloxane, a silicone polyether copolymer or an aminopolysiloxane, provided that the wax and silicone are compatible and that the blend is a solid of melting point 10-200° C. Particularly preferred liquid silicones include polysiloxanes, particularly polydiorganosiloxanes, containing aryl, for example phenyl, or aralkyl, for example benzyl, 2-phenylethyl or 2-phenylpropyl groups in addition to alkyl groups such as methyl. The liquid polydiorganosiloxane can be linear or cyclic; cyclic siloxanes such as tetra(2-phenylpropyl)tetramethylcyclotetrasiloxane may be preferre example be used at up to 100% or even higher, for example up to 200 or 300%, provided that the blend of wax and liquid silicone is solid at 10° C., but is preferably used at 1 to 60%, preferably 10 to 30%, based on the weight of organic wax. An organic liquid, for example liquid paraffin or a naphthenic oil, can be used alternatively or additionally if it is compatible with the blend of fragrance composition and waxy cyclopolysiloxane.

The waxy hydrophobic material can alternatively be a blend of an organic wax with a compatible polysiloxane wax, with or without a liquid silicone. An example of such a blend is a mixture of trimethylsilylated octadecanol with octadecanol, sold under the Trade Mark ‘Dow Corning 580’.

The salt present in the continuous phase can for example be an alkali metal, ammonium or alkaline earth metal salt. It can be an inorganic salt such as a chloride, sulphate or phosphate but is preferably an organic salt, particularly a carboxylate. The salt can be a monocarboxylate such as an acetate or propionate, for example sodium acetate, or a di- or poly-carboxylate salt, for example a succinate, phthalate or citrate. The salt can be a polyelectrolyte, for example a salt of a polymeric acid such as a polycarboxylate, e.g. a polyacrylate or polymethacrylate or a salt of an acrylic or methacrylic acid copolymer. Examples of such polyelectrolyte salts are sold under the Trade Mark ‘Sokolan’.

The salt in the continuous phase can alternatively be a salt of a polycation such as a polymer having pendant quaternary ammonium groups. Examples of such cationic polymers are sold under the Trade Mark ‘Merquat’ and contains dimethyl diallyl ammonium chloride, methacrylamidopropyl trimethyl ammonium chloride or N,N-dimethylimidazolinium chloride groups. Cationically modified cellulose derivatives are also suitable.

The salt preferably has no surfactant properties; in general, the salt should not contain any orgamc group which has a chain of 8 or more carbon atoms unsubstituted by polar groups. The concentration of the salt in the aqueous solution which forms the continuous phase of the emulsion is at least 0.1 M (molar), preferably at least 1 M, up to 5 or 10 M. For some salts, a concentrated aqueous solution can be produced by melting a hydrate of the salt, for example sodium acetate trihydrate, which melts at 58° C. In the case of a salt of a polyelectrolyte, the concentration is at least 0.1 M measured as the concentration of the non-polymeric ion of the salt.

The emulsion can conveniently be formed by melting the blend of wax and perfume and emulsifying it in the continuous phase. Thus according to another aspect of the invention, a process for the preparation of an emulsion containing a fragrance composition in the disperse phase is characterised in that a molten blend of the fragrance composition and a waxy hydrophobic material having a melting point in the range 10-100° C. is emulsified in an aqueous solution of concentration at least 0.1 molar of a salt capable of ionic disassociation in water.

The emulsion can alternatively be made by emulsifying the waxy hydrophobic material in the absence of perfime. The fragrance composition is post-added to the emulsion, which is then heated above the melting point of the waxy hydrophobic material and left standing at this temperature, preferably for a period of at least 10 minutes, for example 30 -60 minutes, allowing the perfume to diffuse within the droplet of hydrophobic waxy material.

The blend of fragrance composition and waxy hydrophobic material is generally emulsified in the salty continuous phase using at least one surfactant. The surfactant is preferably immiscible with the said blend. The surfactant can be a cationic, anionic, nonionic or amphoteric surfactant, although the ionic surfactants are more likely to be immiscible with the perfume wax blend. Cationic surfactants are particularly preferred because of their propensity to adsorb at surfaces, in particular onto fabrics.

Examples of suitable cationic surfactants include alkylamine salts, quaternary ammonium salts, sulphonium salts and phosphonium salts. Examples of suitable anionic organic surfactants include alkali metal soaps of higher fatty acids, alkyl aryl sulphonates, long chain (fatty) alcohol sulphates, olefin sulphates and sulphonates, sulphated monoglycerides, sulphated esters, sulphonated ethoxylated alcohols, sulphosuccinates, alkane sulphonates, phosphate esters, alkyl isethionates, alkyl taurates and/or alkyl sarcosinates. Examples of suitable amphoteric organic detergent surfactants include imidazoline compounds, alkylaminoacid salts and betaines.

According to one aspect of the invention the protected fragrance composition is produced in powdered form, which may be preferred for blending with a solid cleaning product such as a powder detergent. Thus in a process according to the invention for producing a powdered fragrance composition, an emulsion as described above is deposited on a particulate solid carrier.

Examples of suitable solid carriers include soda ash (sodium carbonate), zeolites and other aluminosilicates or silicates, for example magnesium silicate, phosphates, for example powdered or granular sodium tripolyphosphate, sodium sulphate, sodium carbonate, sodium perborate, cellulose derivatives such as sodium carboxymethylcellulose, granulated or native starch and clay.

When the emulsion is to be deposited on a particulate solid carrier, it is preferred that the continuous phase of the emulsion has good coating and binding properties in order to agglomerate the particles of carrier. Emulsions containing a polyelectrolyte salt, particularly a salt of a polymeric acid such as a polyacrylate or polymethacrylate or a salt of an acrylic or methacrylic acid copolymer, generally have good coating and binding properties. Granules with a perfume content of up to 15%, for example 8-12%, by weight can readily be produced by the process of the invention. An emulsion according to the invention can have a perfume content of up to 30 or 40% or even 50% by weight.

The emulsion can for example be sprayed onto the carrier particles and dried subsequently. The particles are preferably-mixed while being treated in a granulation process which produces agglomerated granules. In one preferred process, the particles are agitated in a vertical, continuous high shear mixer in which an emulsion of the fragrance material and the waxy hydrophobic material is sprayed onto the particles. One example of such a mixer is a Flexomix mixer supplied by Hosokawa Schugi. The spraying and mixing produces agglomerated granules. Alternative mixers may be used, for example horizontal mixers such as pin mixers or paddle mixers, ploughshare mixers, twin counter-rotating paddle mixers, or intensive mixers including a high shear mixing arm within a rotating cylindrical vessel. Alternatively a fluid bed coating procedure can be used. Advantageously a process of granulation by mixing can be followed by cooling and drying in a continuous fluid bed.

If needed to improve the granulation process, the emulsion can be diluted with for example water, molten polyethylene glycol or an aqueous solution of polyelectrolyte. If the diluent is not compatible with the original salty continuous phase, the emulsion can comprise particles of perfume wax blend surrounded by the original salty continuous phase dispersed in the diluent. The salty continuous phase will still ensure a protective barrier upon storage, since the perfume and fragrances are poorly soluble in it.

If the continuous phase of the emulsion is an aqueous solution of a polyelectrolyte salt, the granules may be post-coated with a material, for example a polymer, of opposite charge to the polyelectrolyte. If the salt in the continuous phase of the emulsion is a cationic polyectrolyte salt, for example, the granules can be post-coated with an anionic polyelectrolyte. Such post-coating may improve the deposition of the perfume on a fabric which is subsequently washed or rinsed in the presence of the granules.

In an alternative process according to the invention for producing a fragrant powdered cleaning product, the emulsion described above is deposited on a powdered cleaning product, for example by spraying the emulsion onto a detergent powder composition, and is subsequently dried.

In a process according to the invention for producing a fragrant liquid cleaning product, for example a liquid laundry detergent, household cleaning product, fabric softener, hair shampoo or soap or shower gel for personal washing, or a roll-on or spray deodorant, an emulsion as described above is dispersed in the liquid cleaning product. When producing a cleaning product or personal care product in gel form, for example a stick deodorant, an emulsion as described above can be incorporated in the product when it is in liquid form before it is gelled. A tumble drier sheet can be produced by impregnating a textile material with an emulsion as described above.

The emulsion of delayed release fragrance composition according to the invention can alternatively be applied as a coating to a substrate to give sustained release of perfume from the surface.

The invention is illustrated by the following Examples:

### EXAMPLE 1

39.2 g Sokalan (Trade Mark) PA 25 PNCl (a 49% by weight aqueous solution of polyacrylic acid partially neutralised as sodium salt) was mixed with 58.8 g Arquad 16/29 (29% aqueous cationic surfactant solution). Sodium hydroxide was added until a clear homogeneous solution was reached. The solution was heated to 8° C.

64.5 g of a perfume was melt blended with 64.5 g of a waxy hydrophobic polysiloxane of melting point 7° C. which was a linear poly(methyl C30 alkyl)siloxane. The resulting blend was added to the heated polyacrylate solution prepared above and homogenized to form an emulsion. 172.9 g further Sokalan PA 25 PNCl was added to dilute the continuous phase of the emulsion. The emulsion (182.4 g) was then sprayed on sodium carbonate (270 g), granulated in a batch mechanical shear mixer and allowed to dry to form perfumed granules.

### EXAMPLE 2

150 g of the diluted emulsion prepared in Example 1 was sprayed on wessalith XD zeolite (168 g) using the process of Example 1 to form perfumed granules.

### EXAMPLE 3

211.8 g Sokalan CP5 (a 40% aqueous solution of maleic acid-acrylic acid copolymer, sodium salt) was mixed with 66 g Arquad 16/29 and heated to 80° C. 150 g of the 50/50 perfume/polysiloxane wax blend of Example 1 was added and homogenized to form an emulsion. The emulsion was diluted with 108 g water. The diluted emulsion (228 g) was sprayed on wessalith XD zeolite (240 g) using the process of Example 1 to form perfumed granules.

### EXAMPLE 4

55.1 g Sokalan CP5 was mixed with 78.6 g Arquad 16/29 and heated to 8° C. 170 g of the 50/50 perfume/polysiloxane wax blend of Example 1 was added and homogenized to form an emulsion. 196.5 g molten PEG 8000 polyethylene glycol was added and the resulting emulsion (117 g) was then sprayed on wessalith XD (110 g) using the process of Example 1 to form perfumed granules.

The granules of each of Examples 1 to 4 were added to a commercial detergent powder at a level of 0.45% perfume. As a reference, the perfume of Example 1 which had not been encapsulated was added to the same detergent powder at the same perfume loading. Fabrics were washed with each detergent composition in a linitest. The washing conditions are:
Wash volume: 350 ml demin water
Wash load: ca. 11.5 g terry towel
2.1 g concentrated detergent powder
Temperature: 40° C.
Wash time: 30 minutes
After the wash, the fabrics were rinsed twice with 100 ml demin water.

The washed fabrics were submitted to five panelists after drying. Each panelist was confronted with two fabrics washed with the reference and one fabric washed with one of the Example 1 to 4 compositions and was asked to select the most fragrant. The tests were carried out 24, 48 and 72 hours after drying. The following Table 1 reports the number of hits in favour of the granulated sample.

TABLE 1

| Example | After 24 h | After 48 h | After 72 h |
|---|---|---|---|
| 1 | 5 | 5 | 5 |
| 2 | 4 | 4 | 3 |
| 3 | 4 | 4 | 3 |
| 4 | 5 | 5 | 5 |

42.2 g Sokalan CP5 was mixed with 60 g Arquad 16/29 and heated to 8° C. 130 g of the 50/50 perfume/polysiloxane wax blend of Example 1 was added and homogenized to form an emulsion. 150.5 g molten PEG 8000 was added to the emulsion, and the resulting emulsion (167 g) was then sprayed on sodium carbonate (200 g) while mixing in a mechanical shear mixer to form perfumed granules.

The granules of Example 5 were added to a powder detergent as described in Examples 1 to 4 and aged 6 days in the detergent. The resulting detergent composition was then wash tested and assessed as described in Examples 1 to 4. The number of hits in favour of the granulated sample was
After 24 hours—4
After 48 hours—4
After 72 hours—5.

### EXAMPLE 6

25 g Merquat (Trade Mark) 2001 N (polyquaternium-47) were mixed with 13.5 g Arquad 16/29 and heated to 80° C. 50 g of the 50/50 perfime/polysiloxane wax blend of Example 1 was added and homogenized to form an emulsion. 70 g demin water were added and the resulting emulsion was incorporated into a shower gel comprising

| | |
|---|---|
| Sodium laureth sulfate | 10% |
| Decyl glucoside | 2.5% |
| Cocamidopropyl betaine | 3% |
| Laureth-30 | 2% |
| Encapsulated perfume emulsion | 6.25% |
| Water | up to 100% |

4 panellists were treated with two shower gels: one with the non encapsulated perfume and one with the emulsion described above. The perfume intensity on their forearms were evaluated after 1, 2 and 4 hours. In all cases, the encapsulated perfume was preferably chosen.

### EXAMPLE 7

A waxy cyclopolysiloxane of melting point 66° C. was prepared by reacting an olefin mixture consisting predominantly of C26 and C28 olefins with tetramethylcyclotetrasiloxane (cyclic SiH compound).

8 g of cineole, 32 g of the waxy cyclopolysiloxane prepared above, 25 g of a non-surfactant cationic polymer sold under the Trade Mark ‘Merquat 2001 N’ which contains methacrylamidopropyl trinmethyl ammonium chloride groups, 13.5 g of Arquad 16-29 cationic surfactant and 6.0 g of NaCl were weighed in a reactor and heated to 70° C. When the mixture was molten, it was emulsified and diluted with 50 g of water to produce an emulsion having a disperse phase of a blend of cineole and waxy cyclopolysiloxane in an aqueous continuous phase having high ionic strength from the dissolved NaCl and ‘Merquat 2001 N’.

0.42 g of the emulsion was pre-mixed with 1.42 g of fabric softener based on a cationic surfactant and then diluted with 350 mL of soft water. 70 mL of this solution was poured in a Bichner fimnel equipped with a piece of towel as filter (about 3.00 g of towel). The towel was then line dried and the odour intensity was monitored subjectively in a panel test. In a comparative test, the same process was followed using 0.025 g of pure perfume instead of the emulsion. The odour of the sample with free perfume is perceivable during approximately 1.5 hours while the odour of the sample with perfume blended with waxy cyclopolysiloxane is perceivable during about 24 hours.

1. An emulsion comprising a disperse phase which is a blend of a fragrance composition and a waxy hydrophobic material having a melting point in the range 10-200° C. dispersed in a continuous phase comprising an aqueous solution of concentration at least 0.1 molar of a salt capable of ionic disassociation in water.

2. An emulsion according to claim 1 characterised in that the waxy hydrophobic material is a polysiloxane.

3. An emulsion according to claim 2 characterised in that at least 20% of the silicon atoms in the polysiloxane have an alkyl substituent having 16 to 100 carbon atoms.

4. An emulsion according to claim 2 characterised in that the polysiloxane is a cyclic polysiloxane.

5. An emulsion according to claim 1 characterised in that the waxy hydrophobic material is a blend of a wax with a liquid polysiloxane.

6. An emulsion according to claim 5 characterised in that the wax is a waxy non-silicon-containing organic material.

7. An emulsion according to claim 1 characterised in that the aqueous solution is a solution of a polyelectrolyte whose concentration is at least 0.1 molar measured as the concentration of the non-polymeric ion of the salt.

8. An emulsion according to claim 1 characterised in that the salt is an alkali metal carboxylate salt.

9. An emulsion according to claim 1 characterised in that the salt is a salt of a polymer having pendant quaternary ammonium groups.

10. An emulsion according to claim 1 characterised in that the emulsion comprises a surfactant which is not miscible with the blend of fragrance composition and waxy hydrophobic polymer.

11. A process for the preparation of an emulsion as defined in claim 1, characterised in that a molten blend of the fragrance composition and the waxy hydrophobic material having a melting point in the range 10-200° C. is emulsified in an aqueous solution of the salt.

12. A process for the preparation of an emulsion as defined in claim 1, characterised in that the waxy hydrophobic material having a melting point in the range 10-200° C. is emulsified in an aqueous solution of the salt in the absence of the fragrance composition, the fragrance composition is added to the resulting emulsion, and the emulsion is then heated above the melting point of the waxy hydrophobic material and held at a temperature above the melting point of the waxy hydrophobic material to allow the fragrance composition to diff-use within the droplets of waxy hydrophobic material.

13. (canceled)

14. A process for producing a powdered fragrance composition, characterised in that an emulsion according claim 1 is deposited on a particulate solid carrier.

15. A process according to claim 14 in which the continuous phase of the emulsion is an aqueous solution of a polyelectrolyte salt, characterised in that the granules are post-coated with a material of opposite charge to the polyelectrolyte.

16. A process for producing a fragrant powdered cleaning product, characterised in that an emulsion according claim 1 is deposited on a powdered cleaning product.

17. A process according to claim 14 characterised in that the emulsion is deposited by spraying.

18. A process for producing a fragrant liquid cleaning product, characterised in that an emulsion according to claim 1 is dispersed in a liquid cleaning product.

19. (canceled)
